(19) United States
(12) Patent Application Publication
(10) Pub. No.: US 2026/0083325 A1
(43) Pub. Date: Mar. 26, 2026

(54) MULTI-FUNCTIONAL BACKGROUND ILLUMINATOR FOR SLIT LAMPS

(71) Applicants: Clifford Terry, Laguna Beach, CA (US); Kevin William Intrieri, Columbus, OH (US); Andrew Ku, Springfield, OH (US); Ralph Kirkpatrick, Fredericktown, OH (US)

(72) Inventors: Clifford Terry, Laguna Beach, CA (US); Kevin William Intrieri, Columbus, OH (US); Andrew Ku, Springfield, OH (US); Ralph Kirkpatrick, Fredericktown, OH (US)

(21) Appl. No.: 19/339,585

(22) Filed: Sep. 25, 2025

Related U.S. Application Data
(60) Provisional application No. 63/698,972, filed on Sep. 25, 2024.

(51) Int. Cl. A61B 3/135

(52) U.S. Cl. CPC A61B 3/135

(57) ABSTRACT

A multi-functional background illuminator for use with slit lamps is disclosed. The illuminator includes a housing with non-filtered green, blue, and white light-emitting diodes, a rechargeable power supply, an electronic control board, and a rheostat for intensity control. A multi-directional mounting clip attaches the illuminator to a slit lamp tower and permits horizontal and vertical rotation. The green LED enhances visualization of meibomian glands without infrared imaging, the blue LED provides an expanded excitation field for fluorescein dye to improve assessment of tear film and corneal staining, and the white LED supplies wide-field diffused illumination with reduced glare for examination of eyelids, lashes, and conjunctival surfaces. The device extends the diagnostic capabilities of conventional slit lamps by providing wider field illumination and multiple selectable lighting modes in a compact, portable unit.

### I. BACKGROUND

### A. Technical Field

The present invention relates generally to ocular diagnostic equipment, and more particularly to illumination systems for slit lamps that provide improved visualization of ocular structures.

### B. Description of Related Art

Slit lamps are widely used in optometry for examination of the anterior segment of the eye. A slit lamp typically includes a microscope aligned with a narrow, high-contrast slit of light. This arrangement allows for detailed examination of the cornea and lens but is not well suited for broader field visualization of structures such as the lids, lashes, conjunctiva, tear film, lid margins, and meibomian glands.

Conventional approaches to supplementing slit lamp imaging have included infrared cameras for imaging the meibomian glands and fluorescein filters for assessing tear film stability. These approaches suffer from limitations such as high cost, restricted field of view, glare, or inadequate visualization of glandular structures. Accordingly, there is a need for an auxiliary illumination device that can be mounted to existing slit lamps, provide multiple lighting modes, and permit improved diagnostic visualization of ocular tissues.

### II. SUMMARY

The present invention provides a multi-functional background illuminator configured for attachment to conventional slit lamps. The illuminator comprises a compact housing that contains multiple non-filtered light-emitting diodes (LEDs), including green, blue, and white light sources, together with a rheostat, rechargeable power supply, and a multi-directional mounting mechanism.

The green LED provides enhanced visualization of meibomian glands without the need for specialized infrared cameras, as the green light reduces reflections from blood vessels and allows glandular structures to appear in contrast. The blue LED produces a denser excitation spectrum for fluorescein, permitting a wider field (up to 30 mm) for evaluation of tear film dynamics across the entire corneal and conjunctival surface. The white LED provides diffuse, wide-field illumination with reduced glare, aiding examination of the lids, lashes, and mucosal surfaces.

The illuminator is rotatable in both horizontal and vertical directions, allowing the examiner to vary the angle of incidence to reduce glare, minimize shadowing, and improve depth perception. The device is readily attachable to existing slit lamp towers via a swivel clip, thereby enabling operators to extend the diagnostic capabilities of standard slit lamps without the need for costly or specialized cameras.

### III. BRIEF DESCRIPTION OF THE DRAWINGS

The disclosed invention may take physical form in certain parts and arrangement of parts, aspects of which will be described in detail in this specification and illustrated in the accompanying drawings which form a part hereof and wherein:

FIG. 1 depicts a perspective view of a multi-functional background illuminator for slit lamps, showing the housing, LEDs, electronic components, battery, charging port, on/off rheostat switch, and mounting clip.

### IV. DETAILED DESCRIPTION

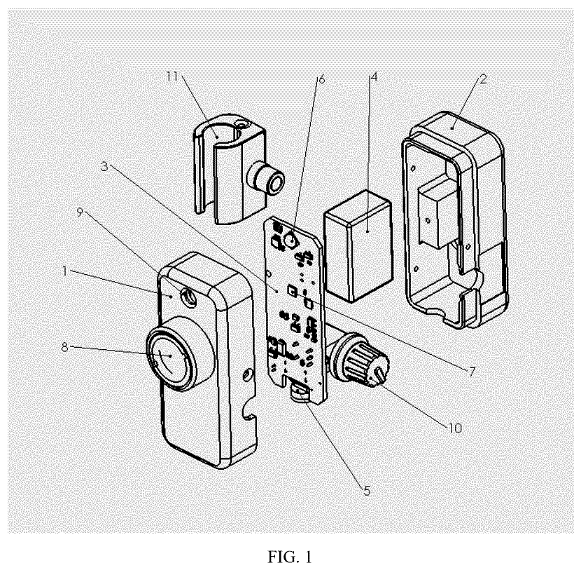

With reference to FIG. 1, the multi-functional background illuminator comprises a front housing (1) and a rear housing (2) enclosing an electronic control board (3), rechargeable battery (4), and charging port (5). The housing supports a plurality of non-filtered LEDs, including a green and white LED (6) and a blue LED (7). The LEDs emit through apertures (8, 9) in the housing. An on/off switch with integrated rheostat (10) is provided for intensity control.

The illuminator is mounted to a slit lamp tower via a multi-directional mounting clip (11). The clip permits horizontal and vertical swivel motion so that the examiner may adjust illumination angles to minimize glare and optimize visualization.

Having thus described the present teaching, it is now claimed:

What is claimed:

1. A background illuminator for a slit lamp, comprising:
a housing containing a plurality of non-filtered light-emitting diodes including a green LED, a blue LED, and a white LED;
an electronic control board and a rechargeable power supply within the housing;
a rheostat coupled to the LEDs for controlling illumination intensity; and
a multi-directional mounting clip configured to attach the housing to a slit lamp and permit horizontal and vertical rotation,
wherein the green LED is configured to provide contrast visualization of meibomian glands, the blue LED is configured to excite fluorescein dye, and the white LED is configured to provide wide-field diffused illumination with reduced glare.
